(19) United States
(12) Patent Application Publication
(10) Pub. No.: US 2010/0249172 A1
(43) Pub. Date: Sep. 30, 2010

(54) 9-CHLORO NOSCAPINE AND ITS USE IN TREATING CANCERS, INCLUDING DRUG-RESISTANT CANCERS

(75) Inventor: Ramesh Chandra, Delhi (IN)

Correspondence Address: INTELLECTUAL PROPERTY / TECHNOLOGY LAW, PO BOX 14329, RESEARCH TRIANGLE PARK, NC, 27709, US

(73) Assignee: EMORY UNIVERSITY, Atlanta, GA (US)

(21) Appl. No.: 12/530,164

(22) PCT Filed: Mar. 4, 2008
(86) PCT No.: PCT/US2008/055818

§ 371 Date: Jun. 2, 2010

Related U.S. Application Data
(60) Provisional application No. 60/904,971, filed on Mar. 5, 2007.

(51) Int. Cl. A61K 31/4355; C07D 491/04; A61P 35/00; A61P 35/02

(52) U.S. Cl. 514/291; 546/90

(57) ABSTRACT

9-Chloro-nos, prodrugs thereof, and pharmaceutically acceptable salts thereof, are disclosed. Pharmaceutical compositions including 9-chloro-nos, and methods of preparation and use thereof are disclosed. 9-Chloro-nos is a noscapine analog that can be used to treat and/or prevent a wide variety of cancers, including drug resistant cancers, by binding tubulin and inducing apoptosis selectively in tumor cells (ovarian and T-cell lymphoma) resistant to paclitaxel, vinblastine and teniposide. 9-Chloro-nos can perturb the progression of cell cycle by mitotic arrest, followed by apoptotic cell death associated with increased caspase-3 activation and appearance of TUNEL-positive cells. Thus, 9-chloro-nos is a novel therapeutic agents for a variety of cancers, including ovarian and T-cell lymphoma cancers, even those that have become drug-resistant to currently available chemotherapeutic drugs.

### FIELD OF THE INVENTION

The present invention relates to the noscapine analog 9-chloro-noscapine, pharmaceutical compositions incorporating the noscapine analog, and methods of using the compound and compositions to treat cancers, including drug resistant cancers.

### BACKGROUND OF THE INVENTION

Microtubules are major cytoskeletal structures responsible for maintaining genetic stability during cell division (Sammak and Borisy, 1987; McIntosh, 1994; Desai and Mitchinson, 1997). The dynamics of these polymers is absolutely crucial for this function that can be described as their growth rate at the plus ends, catastrophic shortening, frequency of transition between the two phases, pause between the two phases, their release from the microtubule organizing center and treadmilling (Margolis and Wilson, 1981; Mitchison and Kirschner, 1984; Kirschner and Mitchison, 1986; Margolis and Wilson, 1998; Jordan and Wilson, 2004). Microtubule lattice also serves as tracks for the axonal transport of organelles driven by anteriograde and retrograde molecular motors to generate and maintain axonal integrity (Joshi, 1998; Nogales, 2000). Interference with microtubule dynamics often leads to programmed cell death and thus microtubule-binding drugs are currently used to treat various malignancies in the clinic (Jordan and Wilson, 2004). Although useful, currently used microtubule drugs such as vincas and taxanes are limited due to the emergence of drug resistance. There have been multiple mechanisms for antimicrotubule drug resistance including overexpression of drug-efflux pumps, misexpression of tubulin isotypes, and perhaps mutational lesions in tubulin itself (Ranganathan et al., 1996; Giannakakou et al., 1997; Monzo et al., 1999; Dumontet et al., 2005).

The pharmacological profile of microtubule-binding agents, however, has not been ideal. Most of them need to be infused over long periods of time in the clinic because they are not water-soluble, and can cause hypersensitive reactions due to the vehicle solution (Rowinsky, 1997). Furthermore, normally dividing cells within the healthy tissues such as intestinal crypts, hair follicles, and the bone marrow are also vulnerable to these agents, leading to toxicities (Rowinsky, 1997). In addition, nerve cells dependent on molecular traffic over long distances undergo degenerative changes causing peripheral neuropathies (Pace et al., 1996; Crown and O'Leary, 2000; Theiss and Meller, 2000; Topp et al., 2000).

Noscapine ((S)-6,7-dimethoxy-3-((R)-4-methoxy-6-methyl-5,6,7,8-tetrahydro[1,3]-dioxolo-[4,5-g]isoquinolin-5-yl)isobenzo-furan-1(3H)-one), a safe antitussive agent for over 40 years, binds tubulin, arrests dividing cells in mitosis and induces apoptosis (Ye et al., 1998). It is well-tolerated in humans and has been shown to be non-toxic in healthy volunteers, including pregnant mothers (Dahlstrom et al., 1982; Karlsson et al., 1990; Jensen et al., 1992).

Unlike the other microtubule-targeting drugs, noscapine does not significantly change the microtubule polymer mass even at high concentrations. Instead, it suppresses microtubule dynamics by increasing the time that microtubules spend in an attenuated (pause) state when neither microtubule growth nor shortening is detectable (Landen et al., 2002). Thus, noscapine-induced suppression of microtubule dynamics, even though subtle, is sufficient to interfere with the proper attachment of chromosomes to kinetochore microtubules and to suppress the tension across paired kinetochores (Zhou et al., 2002a). This represents an improvement over the taxanes, the microtubule-bundling agents or overpolymerizers, and vincas, the depolymerizers, that cause toxicities in mitotic and post mitotic neurons at elevated doses. Noscapine thus effectively inhibits the progression of various cancer types both in cultured cells and in animal models with no obvious side effects (Ye et al., 1998; Landen et al., 2002; Zhou et al., 2002b; 2003; Landen et al., 2004). Surprisingly, the apoptosis is much more pronounced in cancer cells compared with normal healthy cells (Landen et al., 2002).

It would be desirable to have compounds, compositions and methods for preventing and/or treating various types of cancer, without significant associated side effects, that provide increased anti-cancer properties to that of noscapine. The present invention provides such a compound, compositions and methods.

### SUMMARY OF THE INVENTION

9-Chloro-noscapine, prodrugs and metabolites thereof, and pharmaceutically acceptable salts thereof (herein referred to as the “compounds”), and pharmaceutical compositions including the compounds, and methods of preparation and use thereof are disclosed. 9-Chloro-noscapine (9-chloro-nos) is a noscapine analog, with chlorination at the 9-position of the isoquinoline ring. The synthesis, characterization and an evaluation of the anti-tumor potential of 9-chloro-nos is described herein.

9-Chloro-nos binds tubulin, and effectively inhibits cell proliferation of 1A9 (ovarian cancer cells) and its paclitaxel-resistant variant (1A9/PTX22), and human lymphoblastoid cells CEM, and its vinblastine-(CEM/VLB100) and teniposide-(CEM/VM-1-5) resistant variants.

Treatment with 9-chloro-nos selectively halts cell cycle progression at the G2/M phase in cancer cells without affecting the cell cycle of normal human fibroblast cells. This mitotic catastrophe in cancer cells is then followed by induction of apoptosis. The apoptotic mechanism is associated with activation of the key executioner cysteine protease, caspase-3. Most importantly, 9-chloro-nos is more potent against cancer cells that have become resistant to currently used drugs, like vinblastine, teniposide and paclitaxel, as compared to their respective sensitive-parent lines.

The pharmaceutical compositions include an effective amount of the compounds described herein, along with a pharmaceutically acceptable carrier or excipient. When employed in effective amounts, the compounds can act as a therapeutic agent to prevent and/or treat a wide variety of cancers, particularly drug resistant cancers, and are believed to be both safe and effective in this role. Representative cancers that can be treated and/or prevented include drug-resistant ovarian cancer, drug resistant T-cell lymphoma, leukemia, non-small cell lung, colon, central nervous system (CNS), melanoma, renal, ovarian, breast and prostate cancer.

The foregoing and other aspects of the present invention are explained in detail in the detailed description and examples set forth below.

### BRIEF DESCRIPTION OF THE DRAWINGS

FIGS. 1A and B are charts showing the fluorescence quenching of tubulin by 9-Cl-nos. In FIG. 1A, the tubulin fluorescence emission spectrum is quenched by 9-Cl-nos [Control (□), 25 μM (□), 50 μM (▴), 75 μM (□), and 100 μM (□)] in a concentration-dependent manner. FIG. 1B is double reciprocal plot showing a dissociation constant (K_{d}) of 40±8 μM for 9-Cl-nos binding to tubulin. Values are mean±SD for four experiments performed in triplicate (p<0.05). The graphs shown are a representative of four experiments performed.

FIG. 2 is a series of photographs showing the effect of 9-chloro-nos on the morphology of cancer cells. Morphologic criteria for apoptotic cell death include, for example, chromatin condensation with aggregation along the nuclear envelope and plasma membrane blebbing followed by separation into small, apoptotic bodies. Panels show morphological evaluation of nuclei stained with DAPI from control cells (upper panels) and cells treated with 25 μM concentration of 9-Cl-nos for 72 hours (lower panels) using fluorescence microscopy. Several typical features of apoptotic cells such as condensed chromosomes, numerous fragmented micronuclei, and apoptotic bodies are evident (indicated by white arrowheads) upon 72 hours of drug treatment. (Scale bar=15 μm).

FIGS. 3 A-G are a series of charts showing that noscapine and its halogenated analogs inhibit cell cycle progression at mitosis followed by the appearance of a characteristic hypodiploid (sub-G1) DNA peak, indicative of apoptosis. FIGS. 3 A-D depict analyses of cell cycle distribution in a three-dimensional disposition as determined by flow cytometry in MCF-7 cells treated with 25 μM concentration of noscapine and various halogenated noscapine analogues compounds (Nos, 9-F-nos, 9-Cl-nos, 9-Br-nos and 9-I-nos) for 0, 24, 48 and 72 hours respectively. FIGS. 3 E and F show similar three-dimensional profiles for MCF-7 cells treated for 72 hours with 5 μM and 10 μM concentration of each compound to evaluate the differences in percent sub-G1 population among the five compounds. FIG. 4 G is a graphical representation of the quantitation of apoptotic index (percent sub-G1 cells) at the three dose regimes (5 μM, 10 μM and 25 μM) at 72 hours for all compounds. Values and error bars shown in the graph represent the means and standard deviations, respectively of three independent experiments performed in triplicate.

FIG. 4 is a series of immunofluorescence confocal micrographs showing that 9-chloro-nos induce spindles abnormalities. The immunofluorescence confocal micrographs are of MCF-7 cells treated for 0, 12, 24, 48 and 72 hours with a 25 μM concentration of 9-Cl-nos. Mitotic figures are abundant at 24 hours, while apoptotic figures start to appear at 48 hours. (Scale bar=30 μm)

### DETAILED DESCRIPTION OF THE INVENTION

Compounds, pharmaceutical compositions including the compounds, and methods of preparation and use thereof are disclosed.

The following definitions will be useful in understanding the metes and bounds of the invention as described herein.

### I. 9-Chloro-Nos

The compounds include 9-chloro-noscapine (9-Chloro-Nos, (S)-6,7-dimethoxy-3-((R)-4-methoxy-6-methyl-9-chloro-5,6,7,8-tetrahydro-[1,3]dioxolo[4,5-g]isoquinolin-5-yl)isobenzofuran-1(3H)-one)), a noscapine analog chlorinated at the 9-position of the quinoline ring, prodrugs or metabolites of this compound, and pharmaceutically acceptable salts thereof. 9-Chloro-noscapine has the structure shown below.

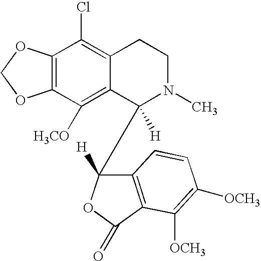

The compound can exist in varying degrees of enantiomeric excess.

The compound can be in a free base form or in a salt form (e.g., as pharmaceutically acceptable salts). Examples of suitable pharmaceutically acceptable salts include inorganic acid addition salts such as sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with an acidic amino acid such as aspartate and glutamate; alkali metal salts such as sodium and potassium; alkaline earth metal salts such as magnesium and calcium; ammonium salt; organic basic salts such as trimethylamine, triethylamine, pyridine, picoline, dicyclohexylamine, and N,N′-dibenzylethylenediamine; and salts with a basic amino acid such as lysine and arginine. The salts can be in some cases hydrates or ethanol solvates. The stoichiometry of the salt will vary with the nature of the components.

### II. Methods of Preparing the 9-Chloro-Nos

9-Chloro-Nos can be prepared by performing electrophilic aromatic substitution on the isoquinoline ring of noscapine, typically under conditions that do not result in significant hydrolysis of the noscapine framework. The chloro substituent can be added to the 9-position on the isoquinoline ring using a variety of known aromatic chlorination conditions, although yields can be optimized and by-products may be present and need to be removed during a purification step. More optimized syntheses are provided in the Examples section.

The halogenation of noscapine involved various procedures, which varied depending on the particular halogen, as summarized below in Scheme 1.

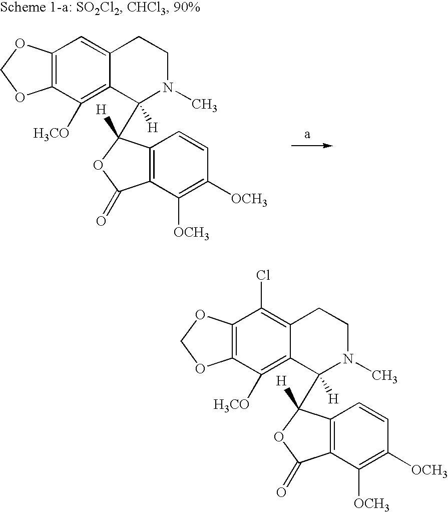

Chlorination of noscapine using sulfuryl chloride in chloroform at low temperature gave excellent yields and the desired regioselectivity.

### III. Pharmaceutical Compositions

The compound, 9-chloro-nos, and its prodrugs and metabolites, and pharmaceutically acceptable salts, as described herein, can be incorporated into pharmaceutical compositions and used to treat or prevent a condition or disorder in a subject susceptible to such a condition or disorder, and/or to treat a subject suffering from the condition or disorder. Optically active compounds can be employed as racemic mixtures, as pure enantiomers, or as compounds of varying enantiomeric purity. The pharmaceutical compositions described herein include 9-chloro-nos, and its prodrugs and metabolites, and pharmaceutically acceptable salts, as described herein, and a pharmaceutically acceptable carrier and/or excipient.

The manner in which the compounds are administered can vary. The compositions are preferably administered orally (e.g., in liquid form within a solvent such as an aqueous or non-aqueous liquid, or within a solid carrier). Preferred compositions for oral administration include pills, tablets, capsules, caplets, syrups, and solutions, including hard gelatin capsules and time-release capsules. Compositions may be formulated in unit dose form, or in multiple or subunit doses. Preferred compositions are in liquid or semisolid form. Compositions including a liquid pharmaceutically inert carrier such as water or other pharmaceutically compatible liquids or semisolids may be used. The use of such liquids and semisolids is well known to those of skill in the art.

The compositions can also be administered via injection, i.e., intraveneously, intramuscularly, subcutaneously, intraperitoneally, intraarterially, intrathecally; and intracerebroventricularly. Intravenous administration is a preferred method of injection. Suitable carriers for injection are well known to those of skill in the art, and include 5% dextrose solutions, saline, and phosphate buffered saline. The compounds can also be administered as an infusion or injection (e.g., as a suspension or as an emulsion in a pharmaceutically acceptable liquid or mixture of liquids).

The formulations may also be administered using other means, for example, rectal administration. Formulations useful for rectal administration, such as suppositories, are well known to those of skill in the art. The compounds can also be administered by inhalation (e.g., in the form of an aerosol either nasally or using delivery articles of the type set forth in U.S. Pat. No. 4,922,901 to Brooks et al., the disclosure of which is incorporated herein in its entirety); topically (e.g., in lotion form); or transdermally (e.g., using a transdermal patch, using technology that is commercially available from Novartis and Alza Corporation). Although it is possible to administer the compounds in the form of a bulk active chemical, it is preferred to present each compound in the form of a pharmaceutical composition or formulation for efficient and effective administration.

Exemplary methods for administering such compounds will be apparent to the skilled artisan. The usefulness of these formulations may depend on the particular composition used and the particular subject receiving the treatment. These formulations may contain a liquid carrier that may be oily, aqueous, emulsified or contain certain solvents suitable to the mode of administration.

The compositions can be administered intermittently or at a gradual, continuous, constant or controlled rate to a warm-blooded animal (e.g., a mammal such as a mouse, rat, cat, rabbit, dog, pig, cow, or monkey), but advantageously are administered to a human being. In addition, the time of day and the number of times per day that the pharmaceutical formulation is administered can vary.

Preferably, the compositions are administered such that active ingredients interact with regions where cancer cells are located. The compounds described herein are very potent at treating these cancers.

In certain circumstances, the compounds described herein can be employed as part of a pharmaceutical composition with other compounds intended to prevent or treat a particular cancer, i.e., combination therapy. In addition to effective amounts of the compounds described herein, the pharmaceutical compositions can also include various other components as additives or adjuncts.

Combination Therapy

The combination therapy may be administered as (a) a single pharmaceutical composition which comprises 9-chloro-nos as described herein, or its prodrugs or metabolites, or pharmaceutically acceptable salts, at least one additional pharmaceutical agent described herein, and a pharmaceutically acceptable excipient, diluent, or carrier; or (b) two separate pharmaceutical compositions comprising (i) a first composition comprising 9-chloro-nos as described herein and a pharmaceutically acceptable excipient, diluent, or carrier, and (ii) a second composition comprising at least one additional pharmaceutical agent described herein and a pharmaceutically acceptable excipient, diluent, or carrier. The pharmaceutical compositions can be administered simultaneously or sequentially and in any order.

In use in treating or preventing cancer, 9-chloro-nos can be administered together with at least one other chemotherapeutic agent as part of a unitary pharmaceutical composition. Alternatively, it can be administered apart from the other anticancer chemotherapeutic agent. In this embodiment, 9-chloro-nos and the at least one other anticancer chemotherapeutic agent are administered substantially simultaneously, i.e. the compounds are administered at the same time or one after the other, so long as the compounds reach therapeutic levels for a period of time in the blood.

Combination therapy involves administering 9-chloro-nos, as described herein, or a pharmaceutically acceptable salt or prodrug of 9-chloro-nos, in combination with at least one anti-cancer chemotherapeutic agent, ideally one which functions by a different mechanism (i.e., VEGF inhibitors, alkylating agents, and the like).

Examples of known anticancer agents which can be used for combination therapy include, but are not limited to alkylating agents, such as busulfan, cis-platin, mitomycin C, and carboplatin; antimitotic agents, such as colchicine, vinblastine, paclitaxel, and docetaxel; topo I inhibitors, such as camptothecin and topotecan; topo II inhibitors, such as doxorubicin and etoposide; RNA/DNA antimetabolites, such as 5-azacytidine, 5-fluorouracil and methotrexate; DNA antimetabolites, such as 5-fluoro-2′-deoxy-uridine, ara-C, hydroxyurea and thioguanine; and antibodies, such as Herceptin® and Rituxan®. Other known anti-cancer agents, which can be used for combination therapy, include arsenic trioxide, gamcitabine, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen and alanosine. Other classes of anti-cancer compounds that can be used in combination with 9-chloro-nosare described below.

9-Chloro-nos can be combined with alpha-1-adrenoceptor antagonists, such as doxazosin, terazosin, and tamsulosin., which can inhibit the growth of prostate cancer cell via induction of apoptosis (Kyprianou, N., et al., Cancer Res 60:4550 4555, (2000)).

Sigma-2 receptors are expressed in high densities in a variety of tumor cell types (Vilner, B. J., et al., Cancer Res. 55: 408 413 (1995)) and sigma-2 receptor agonists, such as CB-64D, CB-184 and haloperidol, activate a novel apoptotic pathway and potentiate antineoplastic drugs in breast tumor cell lines. (Kyprianou, N., et al., Cancer Res. 62:313 322 (2002)). Accordingly, 9-chloro-noscan be combined with at least one known sigma-2 receptor agonists, or a pharmaceutically acceptable salt of said agent.

9-Chloro-nos can be combined with lovastatin, a HMG-CoA reductase inhibitor, and butyrate, an inducer of apoptosis in the Lewis lung carcinoma model in mice, can potentiate antitumor effects (Giermasz, A., et al., Int. J. Cancer 97:746 750 (2002)). Examples of known HMG-CoA reductase inhibitors, which can be used for combination therapy include, but are not limited to, lovastatin, simvastatin, pravastatin, fluvastatin, atorvastatin and cerivastatin, and pharmaceutically acceptable salts thereof.

Certain HIV protease inhibitors, such as indinavir or saquinavir, have potent anti-angiogenic activities and promote regression of Kaposi sarcoma (Sgadari, C., et al., Nat. Med. 8:225 232 (2002)). Accordingly, 9-chloro-noscan be combined with HIV protease inhibitors, or a pharmaceutically acceptable salt of said agent. Representative HIV protease inhibitors include, but are not limited to, amprenavir, abacavir, CGP-73547, CGP-61755, DMP-450, indinavir, nelfinavir, tipranavir, ritonavir, saquinavir, ABT-378, AG 1776, and BMS-232,632.

Synthetic retinoids, such as fenretinide (N-(4-hydroxyphenyl)retinamide, 4HPR), can have good activity in combination with other chemotherapeutic agents, such as cisplatin, etoposide or paclitaxel in small-cell lung cancer cell lines (Kalemkerian, G. P., et al., Cancer Chemother. Pharmacol. 43:145 150 (1999)). 4HPR also was reported to have good activity in combination with gamma-radiation on bladder cancer cell lines (Zou, C., et al., Int. J. Oncol. 13:1037 1041 (1998)). Representative retinoids and synthetic retinoids include, but are not limited to, bexarotene, tretinoin, 13-cis-retinoic acid, 9-cis-retinoic acid, .alpha.-difluoromethylornithine, ILX23-7553, fenretinide, and N-4-carboxyphenyl retinamide.

Proteasome inhibitors, such as lactacystin, exert anti-tumor activity in vivo and in tumor cells in vitro, including those resistant to conventional chemotherapeutic agents. By inhibiting NF-kappaB transcriptional activity, proteasome inhibitors may also prevent angiogenesis and metastasis in vivo and further increase the sensitivity of cancer cells to apoptosis (Almond, J. B., et al., Leukemia 16:433 443 (2002)). Representative proteasome inhibitors include, but are not limited to, lactacystin, MG-132, and PS-341.

Tyrosine kinase inhibitors, such as STI571 (Imatinib mesilate, Gleevec®), have potent synergetic effects in combination with other anti-leukemic agents, such as etoposide (Liu, W. M., et al. Br. J. Cancer 86:1472 1478 (2002)). Representative tyrosine kinase inhibitors include, but are not limited to, Gleevec®, ZD1839 (Iressa®), SH268, genistein, CEP2563, SU6668, SU11248, and EMD121974.

Prenyl-protein transferase inhibitors, such as farnesyl protein transferase inhibitor R115777, possess antitumor activity against human breast cancer (Kelland, L. R., et. al., Clin. Cancer Res. 7:3544 3550 (2001)). Synergy of the protein farnesyltransferase inhibitor SCH66336 and cisplatin in human cancer cell lines also has been reported (Adjei, A. A., et al., Clin. Cancer. Res. 7:1438 1445 (2001)). Prenyl-protein transferase inhibitors, including farnesyl protein transferase inhibitor, inhibitors of geranylgeranyl-protein transferase type I (GGPTase-I) and geranylgeranyl-protein transferase type-II, or a pharmaceutically acceptable salt of said agent, can be used in combination with 9-chloro-nos. Examples of known prenylprotein transferase inhibitors include, but are not limited to, R115777, SCH66336, L-778,123, BAL9611 and TAN-1813.

Cyclin-dependent kinase (CDK) inhibitors, such as flavopiridol, have potent, often synergetic, effects in combination with other anticancer agents, such as CPT-11, a DNA topoisomerase I inhibitor in human colon cancer cells (Motwani, M., et al., Clin. Cancer Res. 7:4209 4219, (2001)). Representative cyclin-dependent kinase inhibitors include, but are not limited to, flavopiridol, UCN-01, roscovitine and olomoucine.

Certain COX-2 inhibitors are known to block angiogenesis, suppress solid tumor metastases, and slow the growth of implanted gastrointestinal cancer cells (Blanke, C. D., Oncology (Huntingt) 16(No. 4 Suppl. 3):17 21 (2002)). Representative COX-2 inhibitors include, but are not limited to, celecoxib, valecoxib, and rofecoxib.

Any of the above-mentioned compounds can be used in combination therapy with the noscapine analogues. Further, 9-chloro-noscan be targeted to a tumor site by conjugation with therapeutically useful antibodies, such as Herceptin® or Rituxan®, growth factors, such as DGF, NGF; cytokines, such as IL-2, IL-4, or any molecule that binds to the cell surface. The antibodies and other molecules will deliver a compound described herein to its targets and make it an effective anticancer agent. The bioconjugates can also enhance the anticancer effect of therapeutically useful antibodies, such as Herceptin® or Rituxan®.

The compounds can also be used in conjunction with surgical tumor removal, by administering the compounds before and/or after surgery, and in conjunction with radiation therapy, by administering the compounds before, during, and/or after radiation therapy.

The appropriate dose of the compound is that amount effective to prevent occurrence of the symptoms of the disorder or to treat some symptoms of the disorder from which the patient suffers. By “effective amount”, “therapeutic amount” or “effective dose” is meant that amount sufficient to elicit the desired pharmacological or therapeutic effects, thus resulting in effective prevention or treatment of the disorder.

When treating cancers, an effective amount of the noscapine analogue is an amount sufficient to suppress the growth of the tumor(s), and, ideally, is a sufficient amount to shrink the tumor, and, more ideally, to destroy the tumor. Cancer can be prevented, either initially, or from re-occurring, by administering the compounds described herein in a prophylactic manner. Preferably, the effective amount is sufficient to obtain the desired result, but insufficient to cause appreciable side effects.

The effective dose can vary, depending upon factors such as the condition of the patient, the severity of the cancer, and the manner in which the pharmaceutical composition is administered. The effective dose of compounds will of course differ from patient to patient, but in general includes amounts starting where desired therapeutic effects occur but below the amount where significant side effects are observed.

The compounds, when employed in effective amounts in accordance with the method described herein, are selective to certain cancer cells, but do not significantly affect normal cells.

For human patients, the effective dose of typical compounds generally requires administering the compound in an amount of at least about 1, often at least about 10, and frequently at least about 25 μg/24 hr/patient. The effective dose generally does not exceed about 500, often does not exceed about 400, and frequently does not exceed about 300 μg/24 hr/patient. In addition, administration of the effective dose is such that the concentration of the compound within the plasma of the patient normally does not exceed 500 ng/mL and frequently does not exceed 100 ng/mL.

IV. Methods of Using the Compounds and/or Pharmaceutical Compositions

The compounds can be used to treat cancers, including blood-borne cancers and solid tumors. Representative cancers include drug-resistant ovarian cancer, drug resistant T-cell lymphoma, leukemia, non-small cell lung, colon, central nervous system (CNS), melanoma, renal, ovarian, breast and prostate cancer.

The compounds can also be used as adjunct therapy in combination with existing therapies in the management of the aforementioned types of cancers. In such situations, it is preferably to administer the active ingredients to in a manner that optimizes effects upon cancer cells, including drug resistant cancer cells, while minimizing effects upon normal cell types. While this is primarily accomplished by virtue of the behavior of the compounds themselves, this can also be accomplished by targeted drug delivery and/or by adjusting the dosage such that a desired effect is obtained without meeting the threshold dosage required to achieve significant side effects.

The following examples are provided to illustrate the present invention, and should not be construed as limiting thereof. In these examples, all parts and percentages are by weight, unless otherwise noted. Reaction yields are reported in mole percentages.

### EXAMPLES

The following examples are provided to illustrate the present invention and should not be construed as limiting the scope thereof. In these examples, all parts and percentages are by weight, unless otherwise noted. Reaction yields are reported in mole percentage.

Chemistry: ¹H NMR and ¹³C NMR spectra were measured by 400 NMR spectrometer in a CDCl₃ solution and analyzed by INOVA. Proton NMR spectra were recorded at 400 MHz and were referenced with residual chloroform (7.27 ppm). Carbon NMR spectra were recorded at 100 MHz and were referenced with 77.27 ppm resonance of residual chloroform. Abbreviations for signal coupling are as follows: s, singlet; d, doublet; t, triplet; q, quartet; m, multiplet. Infrared spectra were recorded on sodium chloride discs on Mattson Genesis II FT-IR. High resolution mass spectra were collected on Thermo Finnigan LTQ-FT Hybrid mass spectrophotometer using 3-nitrobenzyl alcohol or with addition of LiI as a matrix. Melting points were determined using a Thomas-Hoover melting point apparatus and were uncorrected. All reactions were conducted with oven-dried (125° C.) reaction vessels in dry argon. All common reagents and solvents were obtained from Aldrich and were dried using 4 Å molecular sieves. The reactions were monitored by thin layer chromatography (TLC) using silica gel 60 F254 (Merck) on precoated aluminum sheets. Flash chromatography was carried out on standard grade silica gel (230-400 mesh).

### Example 1

### Synthesis of 9-Chloro-Noscapine

### (S)-3-((R)-9-chloro-4-methoxy-6-methyl-5,6,7,8-tetrahydro-[1,3]dioxolo[4,5-g]iso-quinolin-5-yl)-6,7-dimethoxyisobenzofuran-1(3H)-one

To a stirred solution of noscapine (5 g, 12.01 mmol) in chloroform (200 ml), a solution of sulfuryl chloride (4.897 g, 36.28 mmol) in 100 ml chloroform was added drop wise over a period of 1 hour at 5-10° C. The reaction mixture was allowed to attain room temperature and stiffing was continued for 10 hours. The reaction progress was monitored using thin layer chromatography (7% methanol in chloroform). The reaction mixture was poured into 300 ml of water and extracted with chloroform (2×200 ml). The organic layer was washed with brine, dried over anhydrous magnesium sulfate and the solvent evaporated in vacuo to afford the crude product. Purification of the crude product using flash chromatography (silica gel, 230-400 mesh) with 7% methanol in chloroform as an eluent afforded the desired product, (S)-3-((R)-9-chloro-4-methoxy-6-methyl-5,6,7,8-tetrahydro-[1,3]dioxolo[4,5-g]isoquinolin-5-yl)-6,7-dimethoxyisobenzofuran-1(3H)-one (4). Yield: 90% (4.49 g), colorless needles; mp 169.0-169.1° C.; ¹H NMR (CDCl₃, 400 MHz): δ 7.14 (d, 1H, J=8.26 Hz), 6.41 (d, 1H, J=8.26 Hz), 5.93 (s, 2H), 5.27 (d, 1H, J=4.31 Hz), 4.20 (d, 1H, J=4.32 Hz), 3.99 (s, 3H), 3.87 (s, 3H), 3.83 (s, 3H), 2.79-2.65 (m, 2H), 2.54-2.46 (m, 2H), 2.35 (s, 3H); ¹³C NMR (CDCl₃, 100 MHz): δ 167.7, 152.4, 147.5, 139.3, 134.9, 126.1, 120.3, 118.4, 108.5, 102.3, 93.5, 81.9, 64.2, 61.8, 59.6, 57.7, 54.9, 46.1, 45.2, 39.8, 20.6, 18.6; HRMS (ESI): m/z Calcd. for C₂₂H₂₃ClNO₇ (M+1), 448.11481; Found, 448.11482 (M+1).

Other Findings Related to Noscapine Halogenation

Aromatic halogenation constitutes one of the most important reactions in organic synthesis. Although chlorine is extensively used for carrying out electrophilic aromatic substitution reactions in the presence of iron chloride, its utility is limited because of the practical difficulty in handling of these reagents in laboratories compared to N-chlorosuccinimide (NCS). Thus, NCS has proven to be a superior chlorinating reagent provided benzylic chlorination is suppressed. For example, Schmid reported that benzene and toluene gave nuclear brominated derivatives in good yields with NBS (the brominated analogue to NCS) and A1Cl₃ without solvents under long reflux using a large amount of the catalyst (>1 equiv) [30]. However, reactions using NBS in the presence of H₂SO₄, FeCl₃, and ZnCl₂ resulted in relatively low yields (21-61%) together with the polysubstituted products. In another report by Lambert et al., aromatic substituted derivatives were obtained in good yields with NBS in 50% aqueous H₂SO_{4 [}31], however, this method required considerably high acidic conditions which are not suitable for acid labile compounds, such as noscapine. Thus, there still exists a need to develop selective, reproducible and efficient procedures for the halogenation of such labile aromatic compounds that eliminate the limitations associated with the above discussed synthetic methods and offer quantitative yields of the desired compounds. Noscapine consists of isoquinoline and benzofuranone ring systems joined by a labile C—C chiral bond and both these ring systems contain several vulnerable methoxy groups. Thus, achieving selective halogenation at C-9 position without disruption and cleavage of these labile groups and C—C bonds was challenging. After careful titration of many conditions, simple, selective, efficient, and reproducible synthetic procedures have been developed to achieve halogenation at C-9 position. These procedures are discussed below.

Noscapine can be chlorinated using 1:1 equivalents of N-chlorosuccinimide in acetonitrile and ammonium nitrate or ferric chloride as catalyst [34], although this tends to provide relatively low yields. Chlorination of noscapine using sulfuryl chloride in chloroform at low temperature gave excellent yields and the desired regioselectivity. Using this method, 9-Cl-nos (4) was obtained in 90% yield (Scheme 1) [35]. The chlorination took place chemoselectively on ring A of isoquinoline nucleus at position C-9. An absence of C-9 aromatic proton at δ 6.30-ppm in the ¹H NMR spectrum of the product confirmed chlorination at C-9 position. ¹³C NMR and HRMS data support the structure of the compound.

Conclusions:

Relatively simple and straightforward methods for the direct, and regioselective chlorination of noscapine, which provide the chlorinated product in high quantitative yields, are provided herein. Although a plethora of reagents and reaction conditions have been reported for aromatic chlorination, most of them did not work well for noscapine, as it is readily hydrolysable. These synthetic strategies effect the desired transformations under mild conditions.

### Example 4

### Evaluation of Tubulin Binding Properties of Halogenated Noscapine Analogues

Cell Lines and Chemicals:

Cell culture reagents were obtained from Mediatech, Cellgro. CEM, a human lymphoblastoid line was provided by Dr. William T. Beck (Cancer Center, University of Illinois at Chicago). MCF-7 cells were maintained in Dulbecco's Modification of Eagle's Medium 1× (DMEM) with 4.5 g/L glucose and L-glutamine (Mediatech, Cellgro) supplemented with 10% fetal bovine serum (Invitrogen, Carlsbad, Calif.) and 1% penicillin/streptomycin (Mediatech, Cellgro). MDA-MB-231 and CEM cells were grown in RPMI-1640 medium supplemented with 10% fetal bovine serum, and 1% penicillin/streptomycin. Mammalian brain microtubule proteins were isolated by two cycles of polymerization and depolymerization and tubulin was separated from the microtubule binding proteins by phosphocellulose chromatography. The tubulin solution was stored at −80° C. until use.

In Vitro Cell Proliferation Assays

Sulforhodamine B (SRB) assay: The cell proliferation assay was performed in 96-well plates as described previously [12,28]. Adherent cells (MCF-7 and MDA-MB-231) were seeded in 96-well plates at a density of 5×10³ cells per well. They were treated with increasing concentrations of the halogenated analogs the next day while in log-phase growth. After 72 hours of drug treatment, cells were fixed with 50% trichloroacetic acid and stained with 0.4% sulforhodamine B dissolved in 1% acetic acid. After 30 minutes, cells were then washed with 1% acetic acid to remove the unbound dye. The protein-bound dye was extracted with 10 mM Tris base to determine the optical density at 564-nm wavelength.

MTS Assay:

Suspension cells (CEM) were seeded into 96-well plates at a density of 5×10³ cells per well and were treated with increasing concentrations of all halogenated analogs for 72 hours. Measurement of cell proliferation was performed colorimetrically by 3-(4,5-dimethylthiazol-2-yl)-5-(3-carboxymethoxyphenyl)-2-(4-sulphophenyl)-2H-tetrazolium, inner salt (MTS) assay, using the CellTiter96 AQueous One Solution Reagent (Promega, Madison, Wis.). Cells were exposed to MTS for 3 hours and absorbance was measured using a microplate reader (Molecular Devices, Sunnyvale, Calif.) at an optical density (OD) of 490 nm. The percentage of cell survival as a function of drug concentration for both the assays was then plotted to determine the IC₅₀ value, which stands for the drug concentration needed to prevent cell proliferation by 50%.

4′-6-diamidino-2-phenylindole (DAPI) Staining:

Cell morphology was evaluated by fluorescence microscopy following DAPI staining (Vectashield, Vector Labs, Inc., Burlingame, Calif.). MDA-MB-231 cells were grown on poly-L-lysine coated coverslips in 6-well plates and were treated with the halogenated analogs at 25 μM for 72 hours. After incubation, coverslips were fixed in cold methanol and washed with PBS, stained with DAPI, and mounted on slides. Images were captured using a BX60 microscope (Olympus, Tokyo, Japan) with an 8-bit camera (Dage-MTI, Michigan City, Ind.) and IP Lab software (Scanalytics, Fairfax, Va.). Apoptotic cells were identified by features characteristic of apoptosis (e.g. nuclear condensation, formation of membrane blebs and apoptotic bodies).

Tubulin Binding Assay:

Fluorescence titration for determining the tubulin binding parameters was performed as described previously [29]. In brief, 9-Cl-nos (0-100 μM) was incubated with 2 μM tubulin in 25 mM PIPES, pH 6.8, 3 mM MgSO4, and 1 mM EGTA for 45 min at 37° C. The relative intrinsic fluorescence intensity of tubulin was then monitored in a JASCO FP-6500 spectrofluorometer (JASCO, Tokyo, Japan) using a cuvette of 0.3-cm path length, and the excitation wavelength was 295 nm. The fluorescence emission intensity of noscapine and its derivatives at this excitation wavelength was negligible. A 0.3-cm path-length cuvette was used to minimize the inner filter effects caused by the absorbance of these agents at higher concentration ranges. In addition, the inner filter effects were corrected using a formula F corrected ═F observed•antilog [(Aex+Aem)/2], where Aex is the absorbance at the excitation wavelength and Aem is the absorbance at the emission wavelength. The dissociation constant (Kd) was determined by the formula: 1/B=Kd/[free ligand]+1, where B is the fractional occupancy and [free ligand] is the concentration of 9-F-nos, 9-Cl-nos, 9-Br-nos or 9-I-nos. The fractional occupancy (B) was determined by the formula B=ΔF/ΔFmax, where AF is the change in fluorescence intensity when tubulin and its ligand are in equilibrium and ΔFmax is the value of maximum fluorescence change when tubulin is completely bound with its ligand. ΔFmax was calculated by plotting 1/ΔF versus 1/[free ligand].

Cell Cycle Analysis:

The flow cytometric evaluation of the cell cycle status was performed as described previously [12]. Briefly, 2×10⁶ cells were centrifuged, washed twice with ice-cold PBS, and fixed in 70% ethanol. Tubes containing the cell pellets were stored at 4° C. for at least 24 hours. Cells were then centrifuged at 1000×g for 10 min and the supernatant was discarded. The pellets were washed twice with 5 ml of PBS and then stained with 0.5 ml of propidium iodide (0.1% in 0.6% Triton-X in PBS) and 0.5 ml of RNase A (2 mg/ml) for 45 minutes in dark. Samples were then analyzed on a FACSCalibur flow cytometer (Beckman Coulter Inc., Fullerton, Calif.).

Immunofluorescence Microscopy:

Cells adhered to poly-L-lysine coated coverslips were treated with noscapine, 9-chloro-noscapine, and other halogenated analogs (9-F-nos, 9-Br-nos, and 9-I-nos) for 0, 12, 24, 48 and 72 hours. After treatment, cells were fixed with cold (−20° C.) methanol for 5 min and then washed with phosphate-buffered saline (PBS) for 5 min. Non-specific sites were blocked by incubating with 100 μl of 2% BSA in PBS at 37° C. for 15 min. A mouse monoclonal antibody against α-tubulin (DM1A, Sigma) was diluted 1:500 in 2% BSA/PBS (100 μl) and incubated with the coverslips for 2 hours at 37° C. Cells were then washed with 2% BSA/PBS for 10 min at room temperature before incubating with a 1:200 dilution of a fluorescein-isothiocyanate (FITC)-labeled goat anti-mouse IgG antibody (Jackson ImmunoResearch, Inc., West Grove, Pa.) at 37° C. for 1 hour. Coverslips were then rinsed with 2% BSA/PBS for 10 min and incubated with propidium iodide (0.5 μg/ml) for 15 min at room temperature before they were mounted with Aquamount (Lerner Laboratories, Pittsburgh, Pa.) containing 0.01% 1,4-diazobicyclo(2,2,2)octane (DABCO, Sigma). Cells were then examined using confocal microscopy for microtubule morphology and DNA fragmentation (at least 100 cells were examined per condition). Propidium iodide staining of the nuclei was used to visualize the multinucleated and micronucleated DNA in this study.

Results and Discussion

9-Chloro-Noscapine has Higher Tubulin Binding Activity than Noscapine

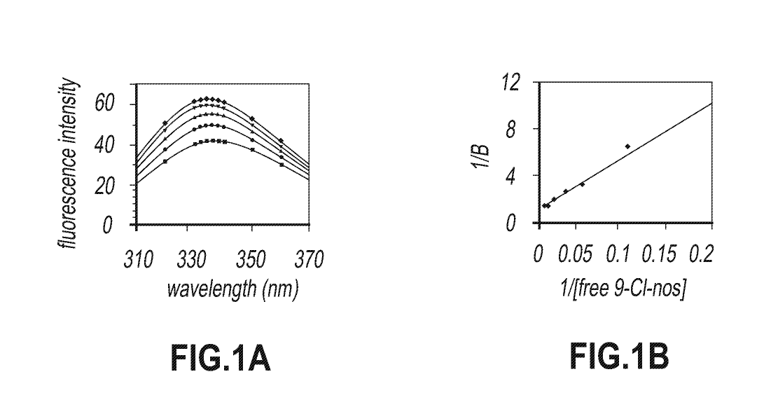

One aspect of the analysis of the anti-tumor properties of the compounds involved determining whether the halogenated noscapine analogs bind tubulin like the parent compound, noscapine. Tubulin, like many other proteins, contains fluorescent amino acids like tryptophans and tyrosines and the intensity of the fluorescence emission is dependent upon the micro-environment around these amino acids in the folded protein. Agents that bind tubulin typically change the micro-environment and the fluorescent properties of the target protein [18,40,41]. Measuring these fluorescent changes has become a standard method for determining the binding properties of tubulin ligands including the classical compound colchicine [42]. This standard method was used to determine the dissociation constant (Kd) between tubulin and 9-chloro-nos. The data showed that 9-chloro-nos quenched tubulin fluorescence in a concentration-dependent manner (FIG. 1A, upper panels). The dissociation constant for noscapine binding to tubulin (Kd) is 144±2.8 μM [18], and 40±8 μM for 9-Cl-nos [43] binding to tubulin. These results thus indicate that 9-chloro-noscapine binds to tubulin with a greater affinity than noscapine.

Effects of 9-chloro-nos on Proliferation of Cancer Cells

Having identified tubulin as the target molecule, the pharmacological study was extended at the cellular level to determine if all the halogenated analogs affected cancer cell proliferation. As a preliminary screen, 9-chloro-nos and noscapine were evaluated for their antiproliferative activity in three human cancer cell lines; human breast adenocarcinoma cells (estrogen- and progesterone-receptor positive, MCF-7 and estrogen- and progesterone-receptor negative, MDA-MB-231) and human lymphoblastoid cells CEM. The compounds were dissolved in DMSO to provide a concentration range of 10 nm to 1000 μM. Sulforhodamine B (SRB) was used in an in vitro proliferation assay to determine the IC₅₀ values that stand for the drug concentration required to achieve a cell kill of 50%. 9-Chloro-nos exhibited potent cytotoxic activity. The IC₅₀ value amounted to 1.9±0.3 μM for MCF-7 cells, which reflects a pronounced antiproliferative activity. A similar low IC₅₀ value of 1.2±0.3 1 μM was measured using 9-chloro-nos for the CEM cells, and 3.5±0.4 μM for MDA-MB-231. Interestingly, the IC₅₀ values of 1.9±0.3 μM and 3.5±0.4 μM with 9-Cl-nos for MDA-MB-231 and MCF-7 cells, respectively, are close, suggesting that 9-Cl-nos inhibits cellular proliferation of cancer cells independent of hormone receptor status. Thus, this preliminary screen with the three chosen cell lines revealed that 9-Cl-nos is a potent cytotoxic compound, exemplified by its much lower IC₅₀ values as compared to noscapine.

Although a definitive correlation of the sensitivity of cancer cells to these analogs cannot yet be established at this stage, it is evident that tubulin represents a potential target for these compounds. The results suggest that the IC₅₀ values do not show a correlation among these analogs and are cell-type dependent.

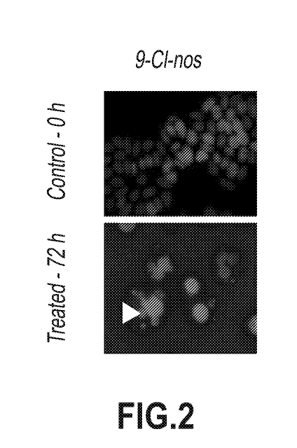

Besides the antiproliferative effect, morphological evaluation using DAPI staining revealed condensed chromatin along with numerous fragmented nuclei (shown by white arrowheads), indicative of apoptotic cell death (FIG. 2), that was investigated next.

Halogenated Noscapine Analogs Alter the Cell Cycle Profile and Cause Mitotic Arrest at G2/M Phase More Actively than Noscapine.

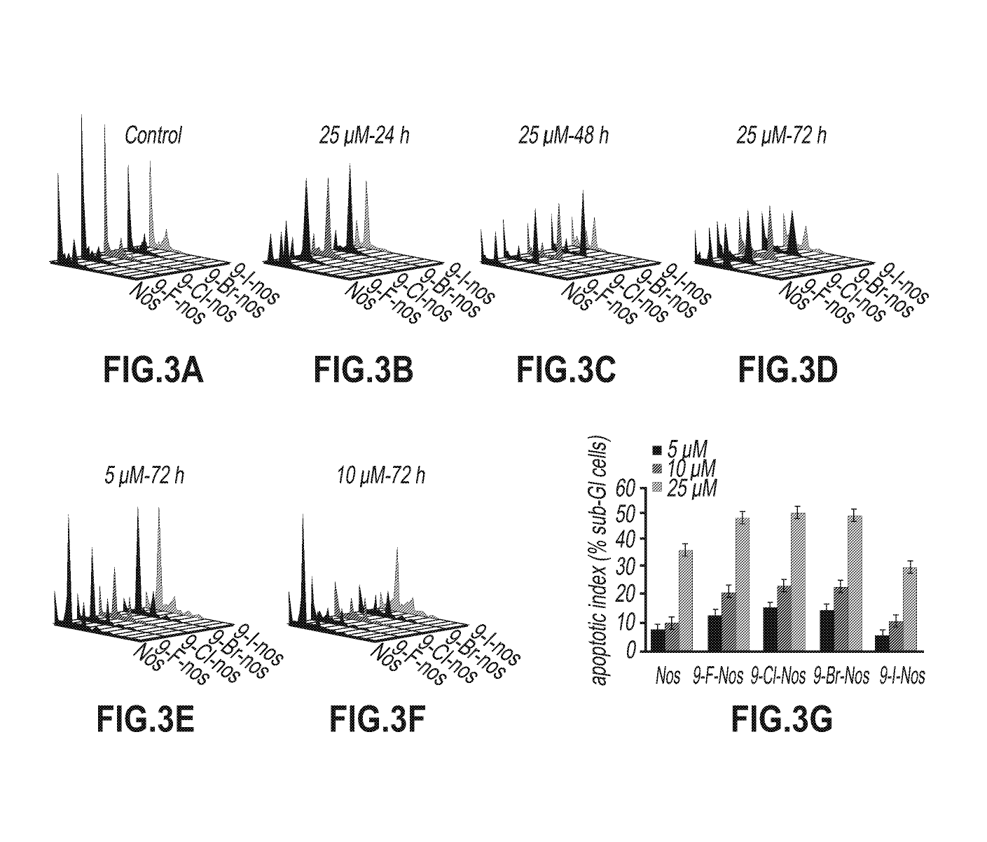

To investigate the precise mechanisms of cell death, the effect of 9-cloro-noscapine and other halogenated noscapine analogs on percent G2/M cells (mitotic index) and percent sub-G1 cells (apoptotic index) was examined as a function of dose and time in MCF-7 cells using fluorescence activated cell sorting (FACS) analysis. The effect of all compounds including noscapine was evaluated at three doses-5 μM, 10 μM and 25 μM for 0, 24, 48 and 72 hours of drug treatment. FIG. 8 (FIGS. 3 A-F) shows the cell cycle profile in a three-dimensional disposition for all the compounds included in the course of this study. Fluorescently labeled DNA is a good indicator of cell cycle progression and cell death. An unreplicated complement of diploid (2N) DNA cells represents the G1 phase while duplicated tetraploid (4N) DNA cells represent G2 and M phases. Cells in the process of DNA duplication between diploid and tetraploid peaks represent S phase when DNA is being synthesized. Less than diploid DNA appears in populations of dying cells that degrade their DNA to different extents. Treatment of MCF-7 cells with these compounds for 0, 24, 48 and 72 hours led to profound perturbations of the cell cycle profile at 25 μM (FIGS. 3 A-D). The results show that 9-F-nos, 9-Cl-nos and 9-Br-nos induced a massive accumulation of cells in the G2/M phase at 24 hours. For example, the G2/M cell population increased from 18.5% in the control to ˜66% in MCF-7 cells treated with 25 μM 9-F-nos for 24 hours. The distribution of cell population over G0/G1, S, G2/M and sub-G1 phases of the cell cycle for 25 μM concentration is shown in Table 1.

TABLE 3
Effect of halogenated derivatives of noscapine on cell cycle progression of MCF-7 cells

| Cell Cycle | | | | | | | | |
|---|---|---|---|---|---|---|---|---|
| parameters | 0 h | | | | 24 h | | | |
| % | Sub-G₁ | G₀/G₁ | S | G₂/M | Sub-G₁ | G₀/G₁ | S | G₂/M |
| Nos | 0.2 ± 0.03 | 54.8 ± 0.03 | 13.2 ± 0.2 | 29.9 ± 2.2 | 10.2 ± 1.2 | 18.3 ± 3.3 | 3.4 ± 0.8 | 50.2 ± 3.6 |
| 9-F-nos | 0.2 ± 0.04 | 67.7 ± 5.6 | 11.9 ± 1.5 | 18.5 ± 2.2 | 6.8 ± 2.1 | 10.8 ± 2.4 | 3.8 ± 1.1 | 65.9 ± 4.4 |
| 9-Cl-nos | 1.4 ± 0.5 | 69 ± 5.6 | 7.5 ± 1.6 | 20.7 ± 3.4 | 15.3 ± 2.5 | 11.5 ± 2.7 | 4.3 ± 1.4 | 59.7 ± 5.2 |
| 9-Br-nos | 0.2 ± 0.1 | 53.3 ± 2.8 | 12.7 ± 1.8 | 28.9 ± 4.4 | 6.9 ± 2.4 | 10.4 ± 1.8 | 3.4 ± 1.2 | 61.9 ± 3.6 |
| 9-I-nos | 0.3 ± 0.2 | 60.6 ± 4.6 | 13.9 ± 2.3 | 23.1 ± 3.7 | 7.7 ± 1.7 | 17 ± 2.3 | 3.1 ± 1.3 | 44.5 ± 5.1 |

| Cell Cycle | | | | | | | | |
|---|---|---|---|---|---|---|---|---|
| parameters | 48 h | | | | 72 h | | | |
| % | Sub-G₁ | G₀/G₁ | S | G₂/M | Sub-G₁ | G₀/G₁ | S | G₂/M |
| Nos | 36.8 ± 3.7 | 19.8 ± 1.9 | 4.1 ± 0.6 | 28.07 ± 0.2 | 36.9 ± 0.2 | 20.5 ± 0.2 | 7.06 ± 0.2 | 26.3 ± 0.2 |
| 9-F-nos | 34.5 ± 2.5 | 7.9 ± 2.8 | 3.5 ± 0.8 | 43.7 ± 2.9 | 48.6 ± 3.3 | 5.2 ± 2.2 | 3.6 ± 1.5 | 34.4 ± 3.4 |
| 9-Cl-nos | 37.8 ± 3.2 | 8 ± 3.3 | 4 ± 1.1 | 42.4 ± 3.8 | 50.2 ± 2.5 | 5.9 ± 1.9 | 4.1 ± 1.4 | 35.3 ± 3.9 |
| 9-Br-nos | 35.1 ± 3.5 | 6.5 ± 2.5 | 2.8 ± 0.7 | 43.8 ± 3.4 | 49 6 ± 2.7 | 4.6 ± 2.1 | 2.4 ± 1.6 | 33.7 ± 3.5 |
| 9-I-nos | 28.1 ± 3.1 | 15.4 ± 4.5 | 2.9 ± 0.6 | 25.9 ± 4.1 | 30.1 ± 3.1 | 17 ± 1.9 | 4.9 ± 2.2 | 20.7 ± 2.2 |

MCF-7 cells were treated with 25 μM solution for the indicated time (h) before being stained with propidium iodide (PI) for cell cycle analysis. Values represent mean ± S.E.M.

More subtle effects that helped us to determine the sensitivity of MCF-7 cells to halogenated analogs for induction of mitotic block were evident at lower dose regimes i.e. 5 and 10 μM. Panels 3 E and F (FIG. 3) show the three-dimensional cell cycle profile of MCF-7 cells treated for 72 hours with 5 μM. and 10 μM, respectively, for all the five compounds. In parallel to the G2/M block, a characteristic hypodiploid DNA content peak (sub-G1) is seen to be rising at 48 and 72 hours of drug treatment for all the three doses studied. The progressive generation of cells having hypodiploid DNA content indicates apoptotic cells with fragmented DNA. The percent sub-G1 population for the three doses (5 μM, 10 μM, and 25 μM) has been plotted for all the compounds in FIG. 3, Panel G. It is evident from the bar-graphical representation that a 72 hour treatment at 25 μM for MCF-7 cells, the percentage of sub-G1 cells is almost similar for 9-F-nos, 9-Cl-nos and 9-Br-nos. However, the sub-G1 population is slightly lower for 9-I-nos than noscapine at 25 μM. At lower doses, the percent sub-G1 cells were higher for 9-F-nos, 9-Cl-nos and 9-Br-nos than the parent compound noscapine. Thus, the data show differences at 5 μM and 10 μM concentrations of the halogenated compounds in the extent of their deleterious effect on the cell cycle by an increase in the percentage of sub-G1 cells having hypodiploid DNA content, characteristic of apoptosis.

Effect of 9-Chloro-Noscapine on Spindle Architecture and Nuclear Morphology

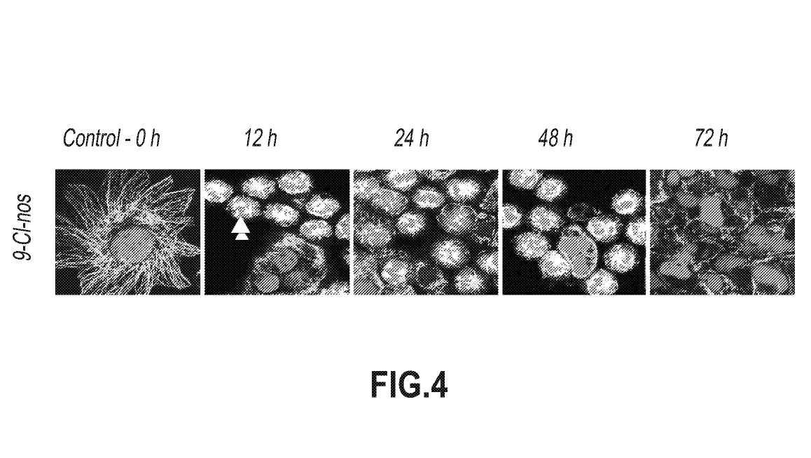

To test whether 9-chloro-noscapine induces spindle abnormalities prior to apoptotic cell death indicated by nuclear fragmentation of dying cells, the spindle architecture and nuclear morphology of MCF-7 cells treated with 9-chloronoscapine was evaluated using confocal microscopy (FIG. 4). MCF-7 cells were treated with 25 μM 9-Cl-nos for 0, 12, 24, 48 and 72 hours. It was found that while untreated MCF-7 cells exhibited normal radial microtubule arrays in normal interphase cells, treated cells exhibited pronounced spindles and condensed chromosomes that are not organized at the metaphase plate indicating mitotic arrest commencing as early as 12 hours, and maximizing at 24 hours of drug treatment, when numerous mitotically arrested cells were visible (indicated by white arrowheads, FIG. 4). While not wishing to be bound to a particular theory, this was probably due to the activation of the spindle assembly checkpoint, a cellular surveillance mechanism that monitors the integrity of the mitotic spindle [20]. The immunofluorescence experiments correlated well with cell cycle progression experiments that offered comparable results at similar time regimens and displayed DNA degradation (sub-G1 population) at 48 and 72 hours of treatment.

Conclusions

Most importantly, the results show that chlorination of noscapine increases its tubulin-binding activity and impacts its therapeutic potential for a variety of cancer cell types. Furthermore, the mechanism of apoptotic cell death caused by these halogenated analogs is preserved, in that, like noscapine, cell death is preceded by extensive mitotic arrest. Taken together, like noscapine, 9-chloro-noscapine indicates a great potential for use as an anti-cancer therapeutic.

### REFERENCES

1. Mitchison T J, Kirschner M. Dynamic instability of microtubule growth. Nature 1984; 312:237-242.
2. Kirschner M, Mitchison T J. Beyond self-assembly: From microtubules to morphogenesis. Cell 1986; 45:329-342.
3. McIntosh J R. The role of microtubules in chromosome movement, in Microtubules 1994 (Hyams J S and Lloyd C W eds) pp 413-434, Wiley-Liss, New York.
4. Jordan M A, Wilson L. Microtubules as a target for anticancer drugs. Nat Rev Cancer 2004; 4:253-265.
5. Zhou J, Giannakakou P. Targeting microtubules for cancer chemotherapy. Curr Med Chem Anticancer Agents 2005; 5:65-71.
6. van Tellingen O, Sips J H, Beijnen J H, Bult A, Nooijen W J. Pharmacology, bio-analysis and pharmacokinetics of the vinca alkaloids and semi-synthetic derivatives. Anticancer Res 1992; 12:1699-1715.
7. Rowinsky E K. The development and clinical utility of the taxane class of antimicrotubule chemotherapy agents. Ann. Rev. Med. 1997; 48:353-374.
8. Crown J, O'Leary M. The taxanes: an update. Lancet 2000; 355:1176-1178.
9. Anderson J T, Ting A E, Boozer S, Brunden K R, Crumrine C, Danzig J, Dent, T. Faga L, Harrington J J, Hodnick W F, Murphy S M, Pawlowski G, Perry R, Raber A, Rundlett S E, Stricker-Krongrad A, Wang J, Bennani Y L. Identification of novel and improved antimitotic agents derived from noscapine. J. Med. Chem. 2005; 48:7096-8.
10. Anderson J T, Ting A E, Boozer S, Brunden K R, Danzig J, Dent T, Harrington J J, Murphy S M, Perry R, Raber A, Rundlett S E, Wang J, Wang N, Bennani Y L. Discovery of S-phase arresting agents derived from noscapine. J Med Chem 2005; 48:2756-2758.
11. Aneja R, Zhou J, Vangapandu S N, Zhou B, Chandra R, Joshi H C. Drug resistant T-lymphoid tumors apoptose selectively by an antimicrotubule agent, EM011. Blood 2006 (In Press, prepublished online Nov. 10, 2005).
12. Zhou J, Gupta K, Aggarwal S, Aneja R, Chandra R, Panda D, Joshi H C. Brominated derivatives of noscapine are potent microtubule-interfering agents that perturb mitosis and inhibit cell proliferation. Mol Pharmacol 2003; 63:799-807.
13. Zhou J, Liu M, Aneja R, Chandra R, Joshi H C. Enhancement of paclitaxel-induced microtubule stabilization, mitotic arrest, and apoptosis by the microtubule-targeting agent EM012. Biochem Pharmacol 2004; 68:2435-2441.
14. Zhou J, Liu M, Luthra R, Jones J, Aneja R, Chandra R, Tekmal R R, Joshi H C. EM012, A microtubule-interfering agent, inhibits the progression of multidrug-resistant human ovarian cancer both in cultured cells and in athymic nude mice. Cancer Chemother Pharmacol 2005; 55:461-465.
15. Checchi P M, Nettles J H, Zhou J, Snyder J P, Joshi H C. Microtubule-Interacting Drugs for Cancer Treatment. Trends Pharmacol Sci 2003; 24:361-365.
16. Joshi H C, Zhou J. Noscapine and Analogues as Potential Chemotherapeutic Agents. Drug News Perspect 2000; 13:543-546.
17. Seetharaman J, Rajan S S. Crystal and Molecular Structure of Noscapine. Zeftschrm fur Kristamographic 1995; 210:111-113.
18. Ye K, Ke Y, Keshava N, Shanks J, Kapp J A, Tekmal R R, Petros J, Joshi H C. Opium alkaloid noscapine is an antitumor agent that arrests metaphase and induces apoptosis in dividing cells. Proc Natl Acad Sci USA 1998; 95:1601-1606.
19. Ye K, Zhou J, Landen J W, Bradbury E M, Joshi H C. Sustained Activation of p34(cdc2) is Required for Noscapine-Induced Apoptosis. J Biol Chem 2001; 276:46697-46700.
20. Zhou J, Panda D, Landen J W, Wilson L, Joshi H C. Minor alteration of microtubule dynamics causes loss of tension across kinetochore pairs and activates the spindle checkpoint. J Biol Chem 2002; 277:17200-17208.
21. Zhou J, Gupta K, Yao J, Ye K, Panda D, Giannakakou P, Joshi H C. Paclitaxel-resistant human ovarian cancer cells undergo c-Jun NH2-terminal kinase-mediated apoptosis in response to noscapine. J Biol Chem 2002; 277:39777-39785.
22. Ke Y, Ye K, Grossniklaus H E, Archer D R, Joshi H C, Kapp J A. Noscapine inhibits tumor growth with little toxicity to normal tissues or inhibition of immune Responses., Cancer Immunol Immunother 2000; 49:217-225.
23. Landen J W, Lang R, McMahon S J, Rusan N M, Yvon A M, Adams A W, Sorcinelli M D, Campbell R, Bonaccorsi P, Ansel J C, Archer D R, Wadsworth P, Armstrong C A, Joshi H C. Noscapine alters microtubule dynamics in living cells and inhibits the progression of melanoma. Cancer Res 2002; 62:4109-4114.
24. Landen J W, Hau V, Wang M S, Davis T, Ciliax B, Wainer B H, Van Meir E G, Glass J D, Joshi H C, Archer D R. Noscapine Crosses the Blood-brain Barrier and Inhibits Glioblastoma Growth. Clin Cancer Res 2004; 10:5187-5201.
25. Dahlstrom B, Mellstrand T, Lofdahl C Johansson M. Pharmacokinetic properties of noscapine. Eur J Clin Pharmacol 1982; 22:535-539.
26. Segal M S, Goldstein M M, Attinger E O. The Use of Noscapine (Narcotine) as an Antitussive Agent. Dis Chest 1957; 32:305-309.
27. Loder R E. Safe Reduction of the Cough Reflex with Noscapine: A Preliminary Communication on a New Use for an Old Drug. Anaesthesia 1969; 24:355-358.
28. Skehan P, Storeng R, Scudiero D, Monks A, McMahon J, Vistica D, Warren J T, Bokesch H, Kenney S, Boyd M R. New colorimetric cytotoxicity assay for anticancer-drug screening. J Natl Cancer Inst 1990; 82:1107-1112.
29. Joshi H C, Zhou J. Gamma tubulin and microtubule nucleation in mammalian cells. Methods Cell Biol 2001; 67:179-193.
30. Schmid H. Bromination with Bromosuccinimide in the Presence of Catalysts. II. Helv Chim Acta 1946; 29:1144-1151.
31. Lambert F L, Ellis W D, Parry R J. Halogenation of Aromatic Compounds by N-Bromo- and N-Chlorosuccinimide under Ionic Conditions. J Org Chem 1965; 30:304-306.
32. Dey B B, Srinivasan T K. Cotamine Series. IV. 5-Bromonarcotine, 5-Bromocotamine, 5-Bromohydrocotamine and 5-Bromonarceine and Their Derivatives. J Indian Chem Soc 1935; 12:526-536.
33. Cainelli G, Manescalchi F. Polymer-Supported Reagents. The Use of Anion-Exchange Resins in the Synthesis of Primary and Secondary Alkyl Fluorides from Alkyl Halides or Alkyl Methanesulfonates. Synthesis 1976; 472-473.
34. Tanemura K, Suzuki T, Nishida Y, Satsumabayashi K, Horaguchi T. Halogenation of Aromatic Compounds by N-chloro-, N-bromo- and N-iodosuccinimide. Chem Lett 2003; 32:932-933.
35. Stokker G E, Deana A A, deSolms S J, Schultz E M, Smith R L, Cragoe E J Jr, Baer J E, Ludden C T, Russo H F, Scriabine A, Sweet C S, Watson L S. 2-(Aminomethyl)phenols, a New Class of Saluretic Agents. 1. Effects of Nuclear Substitution. J Med Chem 1980; 23:1414-1427.
36. Hajipour A R, Arbabian M, Ruoho A E. Tetramethylammonium Dichloroiodate: An Efficient and Environmentally Friendly Iodination Reagent for Iodination of Aromatic Compounds under Mild and Solvent-Free Conditions. J Org Chem 2002; 67:8622-8624.
37. Lee D U. (−)-β-Narcotine: a facile synthesis and the degradation with ethyl chloroformate. Bull Korean Chem Soc 2002; 23:1548-1552.
38. Gleu K, Jagemann W. Action of Iodine Monochloride upon Heterocyclic Bases. J Prakt Chem 1936; 145:257-264.
39. Firouzabadi H, Iranpoor N, Shiri M. Direct and Regioselective Iodination and Bromination of Benzene, Naphthalene and Other Activated Aromatic Compounds Using Iodine and Bromine or Their Sodium Salts in the Presence of the Fe(NO₃)3.1.5N2O4/Charcoal System. Tetrahedron Lett 2003; 44:8781-8785.
40. Peyrot V, Leynadier D, Sarrazin M, Briand C, Menendez M, Laynez J, Andreu J M. Mechanism of binding of the new antimitotic drug MDL 27048 to the colchicine site of tubulin: equilibrium studies. Biochemistry 1992; 31:11125-11132.
41. Panda D, Singh J P, Wilson L. Suppression of microtubule dynamics by LY290181. J Biol Chem 1997; 272:7681-7687.
42. Andreu J M, Gorbunoff M J, Medrano F J, Rossi M, Timasheff S N. Mechanism of colchicine binding to tubulin. Tolerance of substituents in ring C′ of biphenyl analogues. Biochemistry 1991; 30:3777-3786.
43. Aneja R, Lopus M, Zhou J, Vangapandu S N, Ghaleb A, Yao J, Nettles J H, Zhou B, Gupta M, Panda D, Chandra R, Joshi H C. Rational design of a microtubule targeting anti-breast cancer drug, EM015. Cancer Res. (in press)

Having hereby disclosed the subject matter of the present invention, it should be apparent that many modifications, substitutions, and variations of the present invention are possible in light thereof. It is to be understood that the present invention can be practiced other than as specifically described. Such modifications, substitutions and variations are intended to be within the scope of the present application.

1. A compound having the following formula: 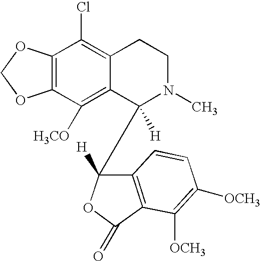
and pharmaceutically acceptable salts thereof.

2. A pharmaceutical composition comprising a compound of claim 1, or a pharmaceutically acceptable salt thereof, and a pharmaceutically acceptable carrier or excipient.

3. A method of treating or preventing cancer in a patient, comprising administering an effective amount of a compound of claim 1 to a patient in need of treatment thereof.

4. The method of claim 16, wherein the cancer is selected from the group consisting of drug-resistant ovarian cancer, drug resistant T-cell lymphoma, leukemia, non-small cell lung, colon, central nervous system (CNS), melanoma, renal, ovarian, breast and prostate cancer.

5. The method of claim 4, wherein the cancer is drug-resistant ovarian cancer or T-cell lymphoma.

6. The method of claim 3, wherein the cancer is a drug-resistant cancer.
